(19) United States
(12) Patent Application Publication
(10) Pub. No.: US 2007/0207199 A1
(43) Pub. Date: Sep. 6, 2007

(54) APPETITE SUPPRESSION DEVICE

(76) Inventor: Stephen J. Sogin, Occidental, CA (US)

Correspondence Address: VIERRA MAGEN MARCUS & DENIRO LLP, 575 MARKET STREET SUITE 2500, SAN FRANCISCO, CA, 94105, US

(21) Appl. No.: 11/617,532

(22) Filed: Dec. 28, 2006

Related U.S. Application Data
(60) Provisional application No. 60/754,391, filed on Dec. 28, 2005.

(51) Int. Cl. A61K 9/48

(52) U.S. Cl. 424/451

(57) ABSTRACT

An appetite suppression device includes an ingestible, expandable membrane in surrounding an expandable reactant material. The membrane is contained in a capsule. A reactant enabling mechanism is integrated with the expandable membrane to enable the reactant after ingestion of the membrane by a subject. enabling mechanism may comprise a pore in one portion of the membrane. Alternatively, the enabling mechanism may be a separation of the membrane into two portions, each having materials which, when combined, react to expand the membrane.

### CLAIM OF PRIORITY

This application claims priority to U.S. Provisional Application No. 60/754,391 entitled “Appetite Suppression Device”, filed Dec. 28, 2005, which is incorporated herein by reference.

### BACKGROUND

The diet industry is a huge business in the United States. People are commonly searching for methods of obtaining weight loss. Safe and effective methods for achieving weight loss are always in need.

Certain prior methods of preventing weight loss have concentrated on percutaneous insertion of balloons and other devices into the stomach. This is costly and uncomfortable for the patient. Convenience and safety are major factors in the success of weight loss programs.

The diet industry extends beyond people. Overweight pets are an increasing concern to many pet owners.

An appetite suppression solution that is effective and easy to use is highly desired.

### SUMMARY

Technology is disclosed for appetite suppression. In one embodiment an appetite suppression device includes an ingestible, expandable membrane in surrounding an expandable reactant material. The membrane is contained in a capsule. A reactant enabling mechanism is integrated with the expandable membrane to enable the reactant after ingestion of the membrane by a subject.

The ingestible appetite suppression device may include an enabling mechanism which comprises a pore in one portion of the membrane. Alternatively, the enabling mechanism may be a separation of the membrane into two portions, each having materials which, when combined, react to expand the membrane.

A method for suppressing appetite in a subject is also disclosed. The method includes determining a dosage comprising a number of appetite suppression devices based on a stomach size of the subject; administering one or more self-expanding appetite suppression devices; and evaluating the subject using an ultrasound test after expansion of the appetite suppression devices.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 depicts an ingestible appetite suppression device.

FIG. 2 depicts a cross-section along line A-A of FIG. 1.

FIG. 3 depicts the appetite suppression device moving through a human subject.

FIG. 4 is a time lapse diagram illustrating the use of an appetite suppression device such as that depicted in FIGS. 1 and 2.

FIG. 5 depicts an exploded region of the pore shown in FIG. 1.

FIG. 6 depicts an alternative embodiment of the appetite suppression device of the technology.

FIG. 7 depicts the appetite suppression device with the enclosure dissolving in the stomach.

FIG. 8 depicts the appetite suppression device expanded in the stomach.

FIG. 9 depicts the appetite suppression device passing through the digestive system.

### DESCRIPTION

Technology is disclosed for suppressing one's appetite. In accordance with the technology, one or more ingestible, self-inflating appetite suppression devices may be provided into the stomach of a subject through the esophageal tube. Once in the stomach, the device inflates for a period of time determined by the construction of the device, and then deflates to be passed by the subject through the digestive track.

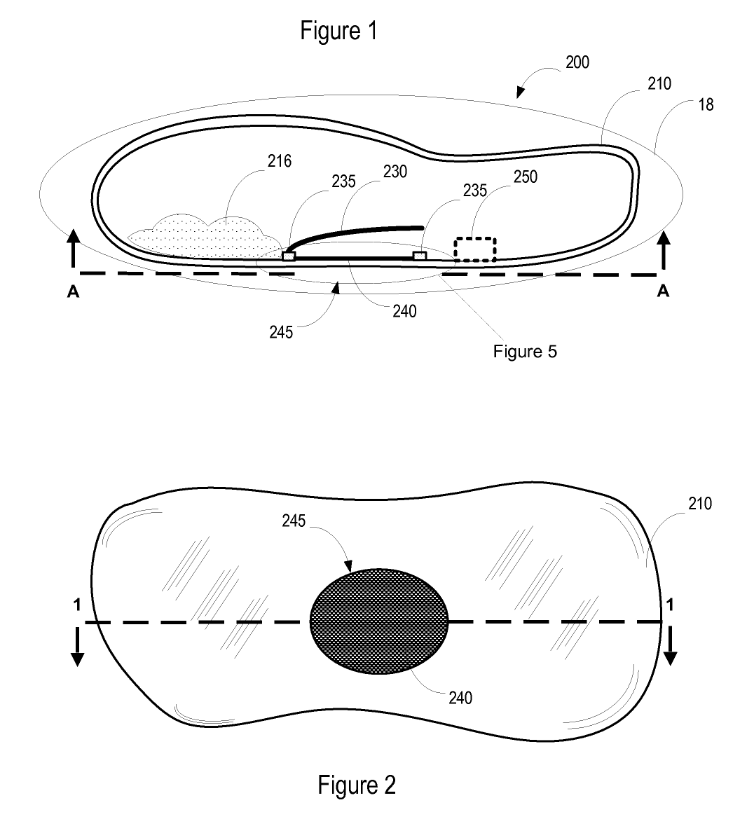

FIGS. 1 and 2 show a first appetite suppression device 200. An appetite suppression device 200 includes semi-permeable gas balloon 210 surrounding a quantity of expanding reactant 216 encapsulated in a dissolving capsule 18 for easy ingestion. Also included in the device 200 is a pore 245 which may include a one-way valve structure 260. As discussed below, the pore is designed to be maintained with an attitude positioned at the bottom of the device. The valve structure may include a mounting ring 235, a mesh plate 240 covering an opening 245 in balloon 210, and a valve 230. In one embodiment, no valve 230 need be provided. If provided, the valve 230 is sized to cover and seal the mesh plate 240. Optionally, one or more weights 250 are also included in the device 200. The weights allow the device 200 to sink into stomach acid present in a subject's stomach and maintains the attitude of the device at an orientation where the pore is positioned at the bottom of the device, in the orientation shown in FIG. 1. The device 200 is shown in a partially inflated form for simplicity, but it should be understood that the balloon may be completely collapsed around the reactant 216, valve structure 230 and weight 250 when encased in capsule 18. In one embodiment, balloon 210 is comprised of a polyurethane plastic material. The material is chosen to have a porosity that allows gas generated by a reaction of stomach acid with reactant 216 to escape from the balloon with the valve structure 230 closed over a period of time. In one case, the porosity is chosen such that enough gas escapes from an inflated balloon to allow passing of the device 200 through the gastrointestinal track after about 24 hours.

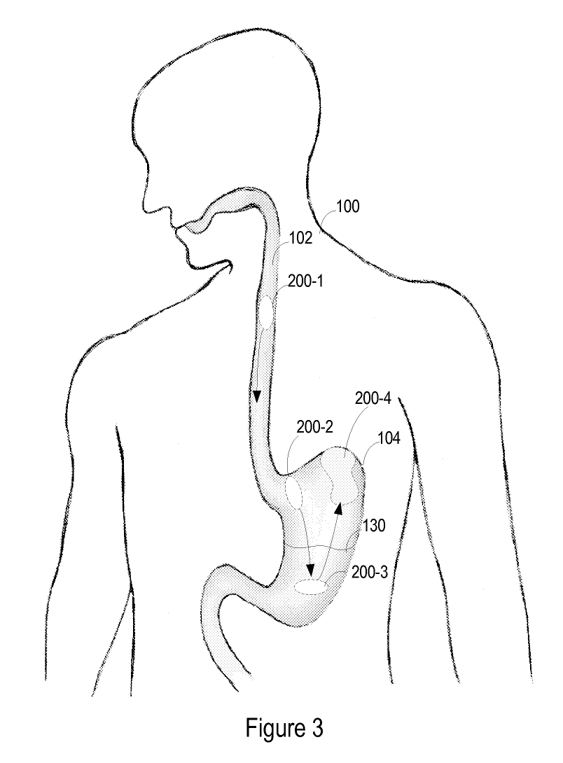

FIG. 3 shows a time-lapse representation of the ingestion of the device in human subject 100 including an esophageal track 103 and stomach 104. To use the device 200, an individual ingests the device (200-1) and the device passes down the track 102 and into the stomach 104 (200-2). The device 200 is activated when the device remains in the stomach (200-3) for a period of time sufficient to dissolve the gel capsule 18. Once the capsule 18 dissolves, the membrane expands by one or more of the mechanisms discussed herein. Expansion by the materials in the device will result in the device expanding in the stomach to cause appetite suppression (200-4). The position of the device in the stomach 104 is exemplary only. The device may position itself anywhere in the stomach.

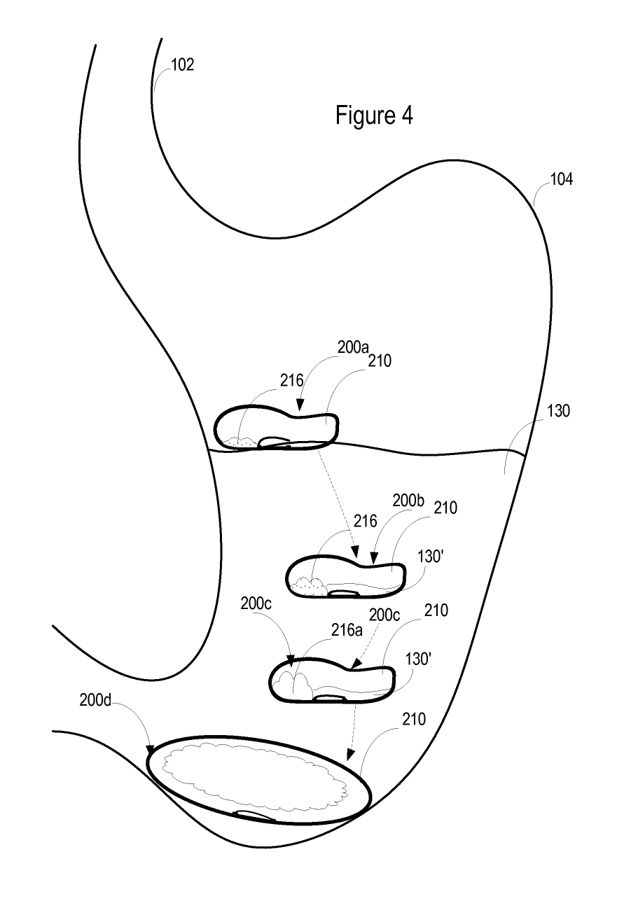

FIG. 4 illustrates the functioning of a single device in a subject 100. FIG. 4 illustrates the progression of a single device 200 through four time-lapse phases. Initially, as shown at 200a, after ingestion the device will come to rest on top of acid 130 in a subject's stomach. The device will naturally orient itself with the ore 245, valve structure 260 and/or weight 250 toward the acid, allowing a portion of the acid to enter the balloon 210. The weight and entrance of acid 130 into the balloon 210 will result in the apparatus 200 sinking into the acid, as shown at 200b. Once acid 130′ enters the balloon, it will begin to react with reactant 216, causing the reactant to generate gas and expand the balloon as shown at 200c and 216a. The expansion of the gas from reactant 216 will cause the valve 230 to cover the mesh structure 240, sealing the balloon and allowing the gas to expand. Nevertheless, the weight of the apparatus will be more significant than the buoyancy provided by the expanding gas, causing the device to rest at the bottom of the stomach. The expanded balloon 200d will rest in the subject for the predetermined period, until expelled in a manner similar to the first embodiment.

The expandable reactant may further comprise a combination of sodium bicarbonate and citric acid, or any reactant which expands in reaction to stomach acid with is generally recognized as safe. In addition, the expandable reactant may be a solid or liquid.

In yet another alternative embodiment, a valve 230 need not be provided to cover the mesh structure 240. It will be understood that because th mesh structure is positioned at the bottom of the apparatus 200 due to the weight of the mesh 240 and ring 230, as well as any added weights 250, the mesh may be configured to allow enough liquid to enter the balloon 210 to generate the reaction, but not allow gas to escape, once inflated. This configuration works because of the inversion of the balloon gas captured with the easiest point of escape (the mesh) positioned at the base of the balloon. Much like air cannot escape when an up-side-down glass is submerged in a tub of water, the gas is trapped in the balloon by the orientation of the gas at the top of the balloon structure.

The mesh structure 240 may comprise any suitable lattice work providing small pore openings sufficient for liquid to pass there through. Structure 240 may be comprised of any medical grade plastic or metal material, with any one or more weights 250 comprised of similar material. In one embodiment, any additional weights 250 are positioned as closely as possible to the mesh structure and opening 245.

In another aspect, a fully inflated device in accordance with any of the embodiments discussed herein may occupy a volume of between 50-500 ml. As noted above, smaller balloons provide greater flexibility in treatment and are easier to ingest, but may require greater numbers in larger patients.

Note that should the balloon become disoriented in the stomach, such that the pore 245 is not at the base of the balloon, gas will escape from the valve allowing deflation of the balloon and allowing the balloon to pass out of the GI tract.

It will be further recognized that multiple balloons may be ingested simultaneously. One advantages of using smaller and multiple devices is that the number of devices used may be more directly adjusted for a particular patient. Smaller and larger stomachs can be accommodated by fewer or more devices, respectively. In such case, the amount material which may be packed into the capsule in the embodiment of FIG. 1 may be limited to an amount which is not sufficient to achieve appetite suppression. In this case, multiple capsules may be ingested.

In one embodiment, a typical dosage of devices comprises four devices, each of which deflates and enters the gastrointestinal tract within twenty-four hours.

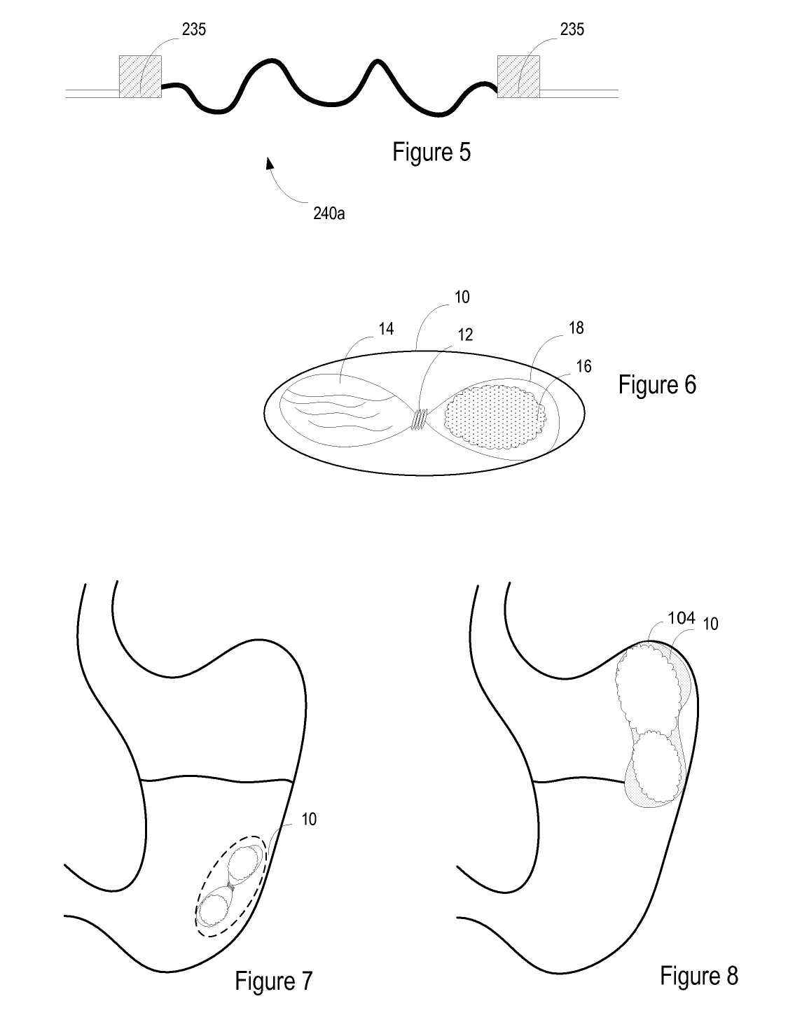

FIG. 5 shows an expanded cross-section of the screen 245 with a further unique feature of the technology. In one embodiment, the screen 245 is manufactured with an uneven surface 240a, which helps ensure that the device cannot become lodged against tissue in any portion of the subject, by creating a surface which tissue cannot bind to. The undulations in the screen 240a help prevent any adhering to tissue by the pore which would result in sealing of the device against the surface.

FIG. 6 shows an alternative appetite suppression device 10 in accordance with a second embodiment the technology. The appetite suppression device 10 includes a gel capsule 18 encasing a twisted balloon 12. One end of the balloon is provided with an reactant, such as water, citric acid or hydrochloric acid 14, while the other end of the balloon includes an expanding reactant such as sodium bicarbonate 16. The state of compression of the twisted balloon 12 within the gel capsule 10 retains the balloon in a twisted configuration and maintains separation between the hydrochloric acid 14 and the sodium bicarbonate 16 sections. While apart, and while contained in the capsule 18, the balloon retains the state shown in FIG. 6. It will be understood that the expandable reactant 16 shown in FIG. 6 may be in a solid form, such as a powder, while the reactant 14 is in a liquid form. In one embodiment, the gel capsule is formed at a size allowing easy ingestion and digestion by a patient.

At rest in the stomach, shown in FIG. 7, the gel capsule 18 will dissolve. As the gel dissolves, the balloon will be free to untwist and the components, such as HCl and bicarbonate, will come into contact with each other. In this example, the reaction of HCl and sodium bicarbonate will form a dioxide (CO₂) and water (H₂O). Any suitable expanding reactants may be used. It will be understood that any liquid acid having an acidity sufficient to cause a reaction with the sodium bicarbonate forming CO₂ may be used in accordance with the technology.

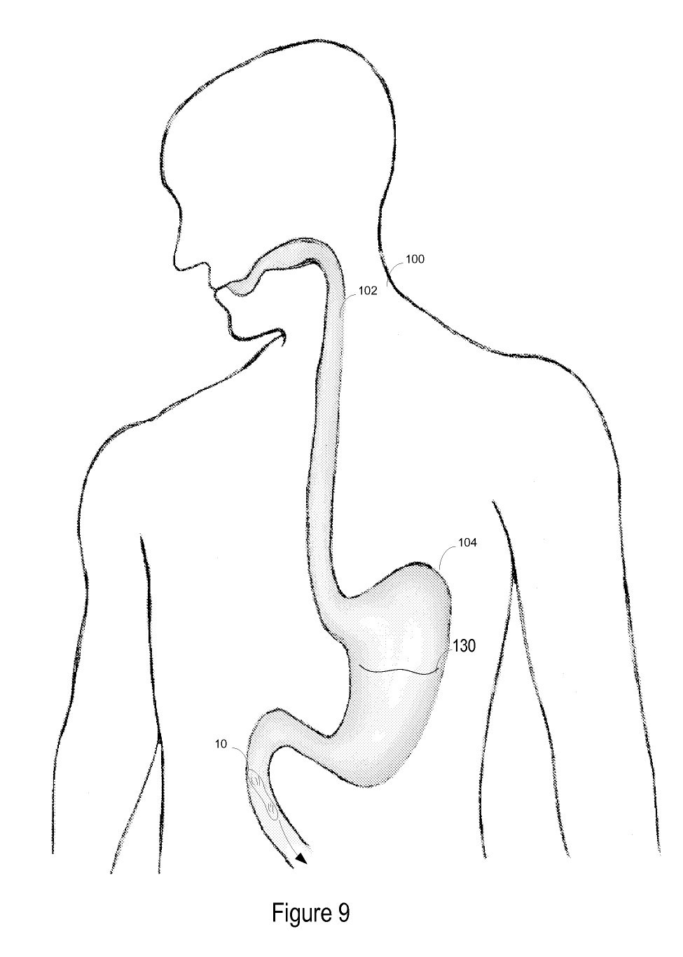

An expanded balloon 18 is shown in FIG. 8. In one embodiment, the inflated balloon occupies 10-80% of a stomach volume. Once the balloon is expanded in the stomach, the expanded balloon will act as an appetite suppressant.

As shown in FIG. 9, in either embodiment, the balloon 10 or 210 will deflate and will pass out the intestinal tract of the individual 100. The balloon can be provided with a chemical which allows deflation after a specific period of inflation, or the individual may ingest a second chemical which deflates the balloon. It will be recognized that the balloon itself will leak gas over time, and the balloon will deflate in the stomach during due to diffusion of the gas through the balloon. It should be recognized the different types of reactants may take the place of HCl 14 and sodium bicarbonate 16. Any acid suitable for generating a reaction with the bicarbonate to generate a gas may be utilized. Any material reacting with an acid or other liquid which generates a gas of sufficient quantity to inflate the balloon may be utilized. The balloon may be made of various materials, including latex, other suitable materials which are resistant to acids present in the stomach. In a further embodiment, the balloon is formed of a material which can easily be recognized by X-ray or other sub-cutaneous visualization techniques.

The technology has specific advantages over mechanical delivery methods for balloons in the stomach. It will further be recognized that other mechanisms may be used to separate the components in the gel capsule, so long as a membrane to the balloon 18 is present in the gel capsule when ingested.

An alternative embodiment of the technology is shown in FIG. 10. In this embodiment, an acid 114 and bicarbonate 116 are carried in dry form in one side of the balloon, while liquid such as water 120 is carried on the other. Water is retained in one side of the balloon by the twisted section 12 between the respective sections. The dry compounds will not react in dry form. Once the gel capsule dissolves, water from the liquid section dissolves the dry components and the reactants create the dioxide (CO₂) which inflates the balloon.

In yet another embodiment, a weak point is provided in the balloon membrane such that excess external pressure on the balloon causes the balloon to rupture. This ensures that should a partially inflated balloon will pass through the digestive system.

In a further embodiment, any of the above designs are created from a material which appears on am ultrasound imaging device. Any number of suitable materials meeting the above functional requirements of the device and which are visible to ultrasound may be utilized. The use of an ultrasound material allows for easy monitoring of the use of the device by a physician. In one example, a physician can determine an initial dosage by making an assessment of the physical size of a subject or a subject's stomach using ultrasound or other techniques. The physician may suggest or prescribe a certain number of devices to be ingested within a time period or until excreted by the subject. Once inside the subject, the physician may use ultrasound to determine the position and volume occupied by the devices relative to the stomach size, and make adjustments in the dosage used by the subject. Repeated checks of the expanded devices may be made.

The devices of the present technology allow multiple treatment techniques to be utilized on multiple different types of subjects.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology and its practical application to thereby enable others skilled in the art to best utilize the technology in various embodiments and with various modifications as are suited to the particular use contemplated. It is intended that the scope of the technology be defined by the claims appended hereto.

I claim:

1. An appetite suppression device, comprising:
a dissolvable capsule;
an expandable membrane in the capsule surrounding an expandable reactant material, the expandable member including an open pore allowing entrance of liquid into the membrane after ingestion of the capsule by a subject.

2. The device of claim 1 wherein the reactant enabling mechanism includes a valve structure positioned in the opening of the membrane to allow stomach acid from the subject to enter the expandable membrane.

3. The device of claim 2 wherein the valve structure includes a mesh cover.

4. The device of claim 3 wherein the valve structure includes a valve cover sealing the mesh cover.

5. The device of claim 1 wherein the expandable reactant is sodium bicarbonate.

6. The device of claim 1 wherein the expandable reactant is a combination of sodium bicarbonate and citric acid.

7. The device of claim 1 wherein the pore is associated with a structure such that the membrane is oriented with the pore at the bottom of the membrane after injection.

8. The device of claim 7 wherein the structure is a weight positioned proximate to the opening.

9. The device of claim 1 wherein the membrane is composed of latex.

10. The device of claim 1 wherein the membrane is composed of polyurethane.

11. The device of claim 1 wherein the membrane has a porosity allowing deflation of an inflated membrane cause by reaction of the expandable reactant within a predefined time period.

12. The device of claim 11 wherein the predefined time period is 24 hours.

13. An ingestible appetite suppression device comprising:
an expandable membrane;
an expandable reactant in the expandable membrane;
a dissolvable container surrounding the membrane and reactant; and
an open pore integrated in the expandable membrane.

14. The device of claim 13 further including a weight adjacent to the pore.

15. The device of claim 13 further including a mesh cover over the pore.

16. The device of claim 13 further including a valve assembly covering the pore upon expansion of the membrane.

17. An appetite suppression device, comprising:
a dissolvable capsule; and
an expandable membrane in the capsule surrounding an expandable reactant material, the expandable membrane including a twisted portion dividing the membrane into first section including the expandable reactant and a second section.

18. The device of claim 17 wherein the second section includes a liquid reactant material.

19. The device of claim 18 wherein the liquid reactant dissolves the expandable reactant to inflate the balloon upon un-twisting of the twisted portion.

20. The device of claim 17 wherein the expandable reactant is sodium bicarbonate.

21. The device of claim 17 wherein the expandable reactant is a combination of sodium bicarbonate and citric acid.

22. The device of claim 17 wherein the membrane is composed of latex.

23. The device of claim 17 wherein the membrane is composed of polyurethane.

24. The device of claim 17 wherein the membrane has a porosity allowing deflation of an inflated membrane cause by reaction of the expandable reactant within a predefined time period.

25. The device of claim 17 wherein the predefined time period is 24 hours.

26. A method for suppressing appetite in a subject, comprising:
determining a dosage comprising a number of appetite suppression devices based on a stomach size of the subject;
administering one or more self-expanding appetite suppression devices; and
evaluating the subject using an ultrasound test after expansion of the appetite suppression devices.

27. The method of claim 26 wherein the step of determining determines a dosage of between one and five devices.

28. The method of claim 26 wherein the step of determining includes using a device having an inflated volume of between 50-500 milliliters.

29. The method of claim 26 wherein the step of administering comprises administering a dissolvable capsule including an expandable membrane in the capsule surrounding an expandable reactant material, the expandable member including an open pore allowing entrance of liquid into the membrane after ingestion of the capsule by a subject.

30. The method of claim 26 wherein the step of administering comprises administering a dissolvable capsule including an expandable membrane in the capsule surrounding an expandable reactant material, the expandable membrane including a twisted portion dividing the membrane into first section including the expandable reactant and a second section.
